(19) United States
(12) Patent Application Publication
(10) Pub. No.: US 2011/0020440 A1
(43) Pub. Date: Jan. 27, 2011

(54) STABLE SOLUTIONS OF SPARINGLY SOLUBLE ACTIVES

(75) Inventors: Indravadan Ambalal Modi, Ahmedabad (IN); Bakulesh Mafatlal Khamar, Ahmedabad (IN); Ashok Sitaram Omray, Ahmedabad (IN); Vandana Patravale, Ahmedabad (IN); Kartik Yogesh Shah, Ahmedabad (IN); Prashant Yogesh Patel, Ahmedabad (IN)

Correspondence Address: Pepper Hamilton LLP, 400 Berwyn Park, 899 Cassatt Road, Berwyn, PA, 19312, US

(73) Assignee: CADILA PHARMACEUTICALS LIMITED, Ahmedabad (IN)

(21) Appl. No.: 12/743,237

(22) PCT Filed: Nov. 15, 2008
(86) PCT No.: PCT/IB2008/003096

§ 371 Date: Sep. 28, 2010

(30) Foreign Application Priority Data
Nov. 19, 2007 (IN) 2281/MUM/2007

(51) Int. Cl. A61K 9/48; A61K 31/167; A61K 31/485; A61P 11/14; A61P 11/02

(52) U.S. Cl. 424/456; 514/629; 514/289

(57) ABSTRACT

The present invention relates to a stable pharmaceutical composition comprising soft gelatin capsules containing at least one sparingly soluble active drug (singly or in combination with sparingly soluble and/or soluble drugs) and a solvent system, wherein the solvent system comprises of solvent, co-solvent, solubilizer(s), surfactant, aqueous solution of alkali and crystal growth inhibitor. The present invention further relates to process for preparing a stable pharmaceutical composition of sparingly soluble active drug(s) in soft gelatin capsules.

### FIELD OF THE INVENTION

The present invention relates to a stable pharmaceutical composition comprising soft gelatin capsules containing atleast one sparingly soluble active drug (singly or in combination with sparingly soluble and/or soluble drugs) and a solvent system, wherein the solvent system comprises of solvent, co-solvent, solubilizer(s), surfactant, aqueous solution of alkali and crystal growth inhibitor. The present invention further relates to process for preparing a stable pharmaceutical composition of sparingly soluble active drug(s) in soft gelatin capsules.

### BACKGROUND OF THE INVENTION

Soft gelatin capsules provide dosage form that are superior to conventional oral dosage forms in terms of improved bioavailability, enhanced drug stability and better patient compliance. Soft gel capsule gets easily dissolved in the stomach and gets absorbed so soft gel capsule offers an attractive means of administering medicaments.

Soft gelatin capsules filled with liquid composition are better than the other liquid oral dosage form because liquid formulation containing medicament may have unacceptable or unpleasant taste. Liquids are encapsulated in the soft gel as solutions or suspensions. Suspensions are pharmaceutically less desirable because they may get settled while manufacturing process, which leads to less uniform product formation. Solution is preferred over suspensions as it provides uniformity to the pharmaceutical active(s). Solution containing more than about 20% water by weight is generally not encapsulated in soft gels, because high water content tends to dissolve the gelatin shell.

Soft gelatin capsules are sensitive to pH, thus the capsules containing concentrated solution of weekly acidic drugs may hydrolyze the gelatin shell, which results into leaking of the capsule. Soft gelatin capsules containing concentrated solution of weekly acidic drug(s) may results into crystallization on long time storage of the dosage form. It may also cause migration of active medicament from the soft gelatin shell.

Pharmaceutical preparations in soft gelatin capsule dosage form are made using Paracetamol alone or in combination with other soluble drug(s) such as Antihistamines like Chlorpheniramine maleate, Doxaylamine succinate; Antitussive like Dextromethorphan HBr; and Decongestants like Pseudoephedrine Hydrochloride, Phenylephrine Hydrochloride.

Sparingly soluble actives pose solubilization problems in soft gelatin capsules containing less volume of the solvent. Acetaminophen (Paracetamol) is the sparingly soluble drug and therefore, it requires relatively large volume of solvent for solubilization. Solubilization of sparingly soluble actives in combination with other pharmaceutical actives is difficult.

The use of large volume of solvent(s) for solubilizing pharmaceutical actives is undesirable because the resulting solution would be so dilute that it requires large dosages form for delivering therapeutically effective amount of active ingredient(s) which is impractical. It would be difficult, to encapsulate such large volume into single gelatin capsules and yet have them be of a reasonable size for easy swallowing.

One of the approaches is to overcome solubility problem by incorporating water, water-miscible co-solvent(s) and/or surfactants) for the formation of pharmaceutical composition. U.S. Pat. No. 4,794,117 discloses the solubilization of hydrophobic pharmaceuticals in aqueous solutions of polyethylene glycol and a surfactant; U.S. Pat. No. 3,784,684 discloses the solubilization of a pharmaceutical active in a mixture of polyethylene glycol and an alcohol having 2-8 carbons and 1-3 hydroxy groups; It is desirable to have poorly soluble drugs like acetaminophen solubilized into a clear solution in as high concentration as possible. It typically involves use of organic solvents.

U.S. Pat. No. 5,484,606 describes the process for reducing the precipitation of difficult to solubilize pharmaceutical actives. It discloses to involve the use of propylene glycol, polyethylene glycol, polyvinyl pyrolidone to achieve solubilization. U.S. Pat. No. 5,510,389 discloses concentrated acetaminophen solution compositions. Use of propylene glycol along with polyethylene glycol and polyvinyl pyrrolidine improves solubility. Both patents may, may not result in a stable pharmaceutical composition.

U.S. Pat. No. 6,287,594 discloses oral liquid compositions with improved bioavailability. They are designed to provide drugs with minimal gastric irritability wherein ratio of active(s) to polymer based dispersing agent is from about 3:1 to 1:50 w/w. The resulting solution is found to be hazy. Polyvinyl pyrrolidone is dispersing agent described. The purpose of invention is not to provide a clear solution.

U.S. Pat. No. 6,383,515 provides a medicament in concentration of 49% to 70% wherein solvent system comprises of low molecular weight polymer and organic acid. It involves heating as a part of process. The process may not result in a clear solution as disclosed in examples.

U.S. Pat. No. 6,387,400 discloses a process for improving concentration of a pharmaceutically active ingredient relative to fill composition. It comprises of two step addition process. In step-1 a suspension of part of a drug is made in polyethylene glycol with a molecular weight of 200 Daltons to 100,000 Daltons and solubilizing it subsequently with hydroxide ion. In step two remaining drug is added and resulting suspension is solubilized by adding remaining part of hydroxide ion. The ratio of a drug to fill material by weight is 1:2 and/or 5:9. The preferred composition is for ibuprofen but the composition using acetaminophen as active is not providing stable pharmaceutical composition.

U.S. Pat. No. 5,919,481 discloses fill material for soft gelatin capsule which is translucent, semisolid in nature. It uses poly alkylene glycol with average molecular weight of about 600 or less along with cellulose ether wherein the compositions as disclosed in US patent '481 are not providing stable pharmaceutical composition.

PCT Publication No. WO 88/02625 discloses the solubilization of an ionized or partly-ionized pharmaceutical active to produce a highly concentrated solution suitable for softgel filling or two piece encapsulation involving the use of polyethylene glycol and water optionally Glycerin or polyvinylpyrrolidone to enhance the solubility of certain drugs wherein pharmaceutical compositions as exemplified in WO 88/02625 such as acetaminophen are not stable.

US Patent Application No. 2004/0157928 A1 discloses pharmaceutical preparations of soft gel capsules using different solvent systems to solubilize sparingly soluble drugs. Solubility is achieved by the use of vehicles such as cation acceptance vehicle, water, surfactants with HLB value 5 to 16, which enhance bioavailability by improving the disintegration degree and dissolution ratio of poorly soluble drug. On the basis of the solubility test mentioned, various pharmaceutical formulations were prepared. However, all the listed formulations do not show satisfactory results.

U.S. Pat. No. 5,071,643 discloses soft gelatin capsules containing concentrated acetaminophen solution comprising 25-40% by weight acetaminophen, hydroxide ions (potassium hydroxide), water and polyethylene glycol. The solvent system involves use of gelling agents like sodium stearate, sodium palmitate and calcium acetate to improve solubility of pharmaceutical ingredients into polyethylene glycol. Moreover, high concentrations of hydroxide ion require to solubilize acetaminophen, which causes increase in pH of the solution resulting degradation of soft gel capsules.

U.S. Pat. No. 5,505,961 describes a method for increasing the solubility of acetaminophen alone or in combination with antihistamines, antitussives, decongestants and expectorants to form clear solutions for encapsulation in soft gel capsules. The compositions comprising acetaminophen, potassium or sodium acetate, polyethylene glycol and polyvinyl pyrrolidone permits 325 mg dose to be administered in the same size soft gel as a 250 mg dosage product. The ratio of polyethylene glycol to polyvinyl pyrrolidine is about 2.5 to 1. It does not involve the use of heat and solvent and/or surfactants. The acetate solvent system does not allow solubilization of other actives in combination with acetaminophen which is the drawback of this system. The compositions as disclosed in US patent '961 are not providing stable pharmaceutical composition.

U.S. Pat. No. 7,029,698B2 discloses an oral pharmaceutical composition in capsular dosage form comprising acetaminophen and lactate salt alone or in combination with acetate salt. The composition exhibit improved solubility of acetaminophen. However, U.S. Pat. No. 7,029,698 doesn't disclose other pharmaceutical actives in combination with acetaminophen. Moreover, the reproducible example mayn't provide stable pharmaceutical composition.

PCT Published Application No. WO 03/013481 discloses a process of manufacturing pharmaceutical composition with increased sparingly soluble pharmaceutical actives in a clear liquid solution filled in soft gelatin capsule. The disclosed concentrated clear liquid pharmaceutical composition comprising 15% to 40% of sparingly soluble pharmaceutical active, 40% to 60% of polyethylene glycol, 4% to 8% of a polyvinyl pyrrolidine and 5% to 10% water. The pharmaceutical compositions as disclosed in WO 03/013481 are not stable and unable to obtain concentrated clear liquid pharmaceutical composition. All the compositions are either not clear or loose their clarity during there shelf life.

It is a long-standing need in the pharmaceutical industry to provide stable pharmaceutical compositions of sparingly soluble active(s) which can be filled in soft gelatin capsules.

### SUMMARY OF THE INVENTION

The main object of the invention is to provide a stable pharmaceutical composition comprising soft gelatin capsules containing sparingly soluble pharmaceutical active(s) and a solvent system.

Another object of the invention is to provide a solvent system capable of producing concentrated solution of atleast one sparingly soluble pharmaceutically active drug suitable for encapsulation in soft gel capsules.

Another object of the present invention is to provide a solvent system for enhancing the solubility of acetaminophen alone or in combination with other pharmaceutical active(s), such as antihistamines like chlorpheniramine maleate, doxaylamine succinate antitussives like dextromethorphan hydrobromide and decongestants like pseudoephedrine hydrochloride, phenylephrine hydrochloride for encapsulation in soft gel capsules.

It is yet another object of the invention is to provide a stable pharmaceutical composition in the form of the solution comprising stable solvent system for filling soft gel capsules, to improve the solubility and stability of the soft gel capsules.

Yet another object of the invention is to provide a solvent system to solubilize the sparingly soluble active(s), wherein the solvent system composed of solvent, solubilizer, co-solvent, surfactant, aqueous alkali solution and crystal growth inhibitor.

Yet another object of the invention is to provide a pharmaceutical composition comprising soft gelatin capsules containing atleast one sparingly soluble active drug (singly or in combination with sparingly soluble and/or soluble drugs) and a solvent system, wherein the solvent system comprises of solvent, co-solvent, solubilizer(s), surfactant, aqueous solution of alkali and crystal growth inhibitor.

### DETAILED DESCRIPTION OF THE INVENTION

A stable pharmaceutical composition in accordance with the present invention provides solubility and stability to sparingly soluble active drug(s) filled in soft gelatin capsules.

A stable pharmaceutical composition in accordance with the present invention wherein the solvent system comprises of solvent, solubilizer, co-solvent, surfactant, aqueous solution of alkali and crystal growth inhibitor to solubilize atleast one sparingly soluble pharmaceutically active drug alone or in combination with other freely soluble active(s).

The soft gel capsules filled with the solution remains stable on long term storage. The solvent system used herein can effectively encapsulate a drug in a highly concentrated solution.

According to the present invention, a pharmaceutical composition comprises medicaments such as sparingly soluble active(s) singly or in combination with other freely soluble active(s). Active drugs are contained in an amount of 25% to 50% of total weight of pharmaceutical composition more preferably 28% to 35% of total weight of pharmaceutical composition.

Sparingly soluble pharmaceutical drug(s) are selected from antipyretic or an analgesic drug, preferably Paracetamol (Acetaminophen), Ibuprofen, Dexibuprofen is used.

Freely soluble pharmaceutical active(s) are selected from antihistamines, antitussives and decongestants; preferably Chlorpheniramine maleate, Doxaylamine succinate, Dextromethorphan HBr and Pseudoephedrine hydrochloride, Phenylephrine hydrochloride are used as medicament.

Solvents used according to the present invention are selected from the group but are not limited to water, benzyl alcohol, ethylene glycol phenyl ether, propylene glycol, propylene glycol phenyl ether, propylene carbonate, phenoxyethanol, dimethyl malonate, dimethyl succinate, diethyl succinate, dibutyl succinate, Transcutol P, dimethyl glutarate, diethyl glutarate, dibutyl glutarate, dimethyl adipate, diethyl adipate, dibutyl adipate, various grades of polyethylene glycol or mixtures thereof. Transcutol P (Diethylene glycol mono ethyl ether), is selected as solvent for the preparation of stable solution in an amount of 20 to 60% of the total weight of dosage form more preferably in an amount of 33 to 38% of the total weight of the pharmaceutical composition. Transcutol-P may be useful as solvent and/or surfactant for the preparation of pharmaceutical composition of poorly soluble active(s).

Co-solvent is selected from polyethylene glycol (PEG) having molecular weight from about 200 to about 6000 Daltons. The most preferred embodiment of invention uses polyethylene glycol having a molecular weight of about 400 Dalton. The solvent may comprise combinations of PEGs, preferably 400 and 1000 Dalton, to enable the active medicament remain sustained in the solubilized form. Binary use of PEGs in the solvent system avoids the frequent problem of leaking of the gelatin shell. In one of the embodiment, PEG(s) are combined wherein atleast one PEG is below 800 Dalton and atleast one PEG is above 800 Dalton. The co-solvent is used in an amount ranging from 5% to 21% of the total weight of pharmaceutical composition.

Solubilizers selected for solubilization of the sparingly soluble active(s) are Polyvinyl pyrrolidone having k value from 10 to 120, Macrogol 15 Hydroxystearate (Solutol), Propylene Glycol Caprylate (Capryol) and Polyoxyl 40 Hydrogenated Castor Oil (Acrysol). Preferably Polyvinyl pyrollidone (PVP) is used. The solubilizer(s) is used in an amount ranging from used in an amount 5 to 15% of the total weight of the pharmaceutical composition.

Sodium Hydroxide and Potassium Hydroxide are used for preparing alkali solution. Preferably aqueous Sodium hydroxide (NaOH) is used to adjust the pH of the solution containing weekly acidic drug(s). The amount of NaOH used is 0% to 0.5% of the total weight of pharmaceutical composition. Water is used in an amount 0 to 7% of the total weight of pharmaceutical composition. High concentration of hydroxide ion may lead to breakage of soft gelatin capsule shell. In one embodiment, the hydroxide ion is present in the instant invention in an amount between 0.1 to 1.0 moles of hydroxide ion per mole of active ingredient(s). In another embodiment, the hydroxide ion is present in the instant invention in an amount between 0.0001 to 0.1 moles of hydroxide ion per mole of active ingredient(s).

Surfactant(s) are selected from Sodium Lauryl Sulphate (SLS), Propylene Glycol Caprylate (Capryol 90), Polysorbate 20 (Tween 20), Diethylene glycol mono ethyl ether (Transcutol P), Polysorbate 80 (Tween 80), Gelucire 44/14. The preferred surfactant used is Diethylene glycol mono ethyl ether (Transcutol P). HLB value of the selected surfactants is ranging from 1-40.

Crystal growth inhibitors selected in the present invention are Polyvinyl Alcohol, Gelatin, Mannitol, Sodium Carboxymethyl Cellulose (CMCNa) and Povidone (Polyvinyl pyrrolidone—PVP). The preferred crystal growth inhibitor used is Povidone. Crystal growth inhibitor can be used in an amount 5 to 15% of the total weight of the pharmaceutical composition.

The size of capsules can vary in accordance to the volume of the solution intended to be contained therein.

The soft gel capsules encapsulated with the solution containing solvent system and sparingly soluble active(s) in combination with other freely soluble active(s), is further analysed for stability. The soft gelatin capsules were found to be stable chemically and physically on long term storage with improved shelf life.

In accordance with the present invention, preferred embodiments includes gelatin in the range of about 40% to 48% and a plasticizer ranging in amount from about 5% to 35%. Another preferred plasticizer is sorbitol BP, a non-crystallizing sorbitol solution. When either a 70%, non-crystallizing sorbitol solution or Anidrisorb 85/70 are used alone, the amount of plasticizer used preferably ranges from about 10% to 35%. Capsule formulations can also include other suitable additives, for example coloring agents, which impart specific characteristics such as the look and feel of the capsule. FD&C dyes and D & C dyes are examples of pharmaceutically acceptable coloring agents that may be used in preferred embodiments.

The term stable as used herein disclose that the active drug(s) did not precipitate in soft gelatin capsules for shelf life of the product.

The general process of preparation of stable soft gelatin capsule dosage form of sparingly soluble pharmaceutical actives comprising steps of a) solubilization of pharmaceutical actives in solvent system comprising of solvent, solubilizer, co-solvent, surfactant, crystal growth inhibitor and aqueous alkali solution. b) encapsulation of pharmaceutical active solution in soft gelatin shell dosage form. In another embodiment, the process of preparation of stable soft gelatin capsule dosage form of sparingly soluble pharmaceutical actives comprising steps of a) Mixing of Transcutol P and polyethylene glycol (s) under constant stirring with heating. b) Dissolve polyvinyl pyrollidone in the above solvent blend under constant stirring with heating. c) Dissolve Acetaminophen and optionally other active drugs in the above solvent system with heating not more than 65° C. under constant stirring. Adjust the pH of the solution in the range of 7-7.4 by adding 1% sodium hydroxide solution.

Encapsulate the concentrated solution in the soft gelatin capsules.

The following examples are intended to demonstrate some embodiments of the invention without limiting the scope of the invention.

### EXAMPLES

The following examples though solubilize paracetamol; they don't provide stable pharmaceutical composition in a soft gel cap.

### Example 1

TABLE 1
Composition of example 1

| Ingredients | Mg/Capsule |
|---|---|
| Paracetamol | 350.00 |
| Transcutol P | 465.00 |
| PEG 400 | 175.00 |
| PVP K30 | 100.00 |
| Purified Water | 40.00 |
| NaOH | 0.50 |
| Total weight | 1130.50 |

A solution was prepared to fill in a batch of 5000 soft gel capsules.

2.325 kg Transcutol P and 875 gm polyethylene glycol 400 were mixed and heated with constant stirring. 500 gm of polyvinyl pyrollidone K-30 was dissolved in the above solvent and heated with constant stirring. Acetaminophen 1750 gm was dissolved in the above solvent system with heating not more than 60° C. under constant stirring. The pH of the solution was adjusted from 6.5 to 7.2 by adding 1% sodium hydroxide solution. The concentrated solution was encapsulated in the soft gelatin capsules.

### Example 2-6

TABLE 2
Composition of examples 2-6

| | Mg per Capsule | | | | |
|---|---|---|---|---|---|
| Ingredients | Example 2 | Example 3 | Example 4 | Example 5 | Example 6 |
| Paracetamol | 325.00 | 325.00 | 325.00 | 325.00 | 325.00 |
| Pseudoephedrine HCl | 30.00 | 30.00 | 30.00 | 30.00 | 30.00 |
| Transcutol P | 270.00 | 75.00 | 75.00 | 75.00 | 70.00 |
| PEG 400 | 405.00 | 515.00 | 525.00 | 555.00 | 555.00 |
| PEG 6000 | — | 30.00 | 30.00 | — | — |
| PVP K 30 | — | 55.00 | 55.00 | 55.00 | — |
| Solutol | — | — | — | — | 75.00 |
| Total wt. | 1030.00 | 1030.00 | 1040.00 | 1040.00 | 1055.00 |
| Observation | Turbid | Crystal | Initially clear, | Crystal | Turbid |
| | solution | formation | After one week | formation | solution |
| | | on cooling | crystal formation | on cooling. | |
| | | | was observed. | | |

| | |
|---|---|
| Remarks | Unstable Compositions |

### Example 7

TABLE 3
Composition of example 7

| Ingredients | Mg/Capsule |
|---|---|
| Paracetamol | 400.00 |
| PEG 400 | 530.00 |
| PEG 1000 | 30.00 |
| PVP K30 | 55.00 |
| Purified Water | 75.00 |
| Total weight | 1090.00 |

A solution was prepared to fill in a batch of 20,000 soft gel capsules.

10.6 Kg polyethylene glycol 400, 1.5 Kg purified water, and 0.6 Kg polyethylene glycol 1000 were mixed and heated with constant stirring. 1.1 Kg polyvinyl pyrollidone K-30 was dissolved in the above solvent with constant stirring. 8.0 Kg Acetaminophen was dissolved in the above solvent system with heating under constant stirring. The concentrated solution was encapsulated in the soft gelatin capsules.

### Example 8

TABLE 4
Composition of example 8

| Ingredients | Mg/Capsule |
|---|---|
| Paracetamol | 400.00 |
| PEG 400 | 530.00 |
| PEG 1000 | 35.00 |
| PVP (K30 + K90) | 57.50 + 27.50 |
| Purified Water | 75.00 |
| Total weight | 1125.00 |

A solution was prepared to fill in a batch of 20,000 soft gel capsules.

10.6 Kg polyethylene glycol 400, 1.5 Kg purified water, and 0.7 Kg polyethylene glycol 1000 were mixed and heated with constant stirring. 1.1 Kg polyvinyl pyrollidone (PVP K-30 & PVP K-90) were dissolved in the above solvent with constant stirring. 8.0 Kg Acetaminophen was dissolved in the above solvent system with heating under constant stirring. The concentrated solution was encapsulated in the soft gelatin capsules.

### Example 9

TABLE 5
Composition of example 9

| Ingredients | Mg/Capsule |
|---|---|
| Paracetamol | 400.00 |
| PEG 400 | 530.00 |
| PEG 1000 | 30.00 |
| PVP K90 | 55.00 |
| Purified Water | 80.00 |
| Total weight | 1095.00 |

A solution was prepared to fill in a batch of 20,000 soft gel capsules.

10.6 Kg polyethylene glycol 400, 1.6 Kg purified water, and 0.6 Kg polyethylene glycol 1000 were mixed and heated with constant stirring. 1.1 Kg polyvinyl pyrollidone was dissolved in the above solvent with constant stirring. 8.0 Kg Acetaminophen was dissolved in the above solvent system with heating under constant stirring. The concentrated solution was encapsulated in the soft gelatin capsules.

### Example 10

TABLE 6
Composition of example 10

| Ingredients | Mg/Capsule |
|---|---|
| Paracetamol | 450.00 |
| PEG 400 | 550.00 |
| PEG 1000 | 30.00 |
| PVP K90 | 60.00 |
| Purified Water | 85.00 |
| Total weight | 1175.00 |

A solution was prepared to fill in a batch of 20,000 soft gel capsules.

11.0 Kg polyethylene glycol 400, 1.7 Kg purified water, and 0.6 Kg polyethylene glycol 1000 were mixed and heated with constant stirring. 1.2 Kg polyvinyl pyrollidone was dissolved in the above solvent with constant stirring. 9.0 Kg Acetaminophen was dissolved in the above solvent system with heating under constant stirring. The concentrated solution was encapsulated in the soft gelatin capsules.

### Example 11

TABLE 7
Composition of example 11

| Ingredients | Mg/Capsule |
|---|---|
| Paracetamol | 400.00 |
| PEG 400 | 520.00 |
| PEG 1000 | 30.00 |
| PVP K30 | 90.00 |
| Purified Water | 75.00 |
| Potassium Acetate | 6.00 |
| Total weight | 1121.00 |

A solution was prepared to fill in a batch of 20,000 soft gel capsules.

10.4 Kg polyethylene glycol 400, 1.5 Kg purified water, and 0.6 Kg polyethylene glycol 1000 were mixed and heated with constant stirring. 1.8 Kg polyvinyl pyrollidone and 6.0 Kg potassium acetate were dissolved in the above solvent with constant stirring. 8.0 Kg Acetaminophen was dissolved in the above solvent system with heating under constant stirring. The concentrated solution was encapsulated in the soft gelatin capsules.

### Example 12

TABLE 8
Composition of example 12

| Ingredients | Mg/Capsule |
|---|---|
| Paracetamol | 450.00 |
| PEG 400 | 400.00 |
| PEG 200 | 120.00 |
| PEG 1450 | 44.00 |
| PVP K30 | 90.00 |
| Purified Water | 75.00 |
| Total weight | 1179.00 |

A solution was prepared to fill in a batch of 20,000 soft gel capsules.

Polyethylene glycol 400, Polyethylene glycol 200, Polyethylene glycol 1450 and purified water were mixed and heated with constant stirring. Polyvinyl pyrollidone was dissolved in the above solvent with constant stirring. Paracetamol was dissolved in the above solvent system with heating under constant stirring. The concentrated solution was encapsulated in the soft gelatin capsules.

### Example 13

TABLE 9
Composition of example 13

| Ingredients | Mg/Capsule |
|---|---|
| Paracetamol | 400.00 |
| PEG 400 | 483.00 |
| PEG 1500 | 29.00 |
| PVP K90 | 63.00 |
| Purified Water | 175.00 |
| Total weight | 1217.00 |

A solution was prepared to fill in a batch of 20,000 soft gel capsules.

0.966 Kg polyethylene glycol 400, 3.5 Kg purified water, and 0.58 Kg polyethylene glycol 1500 were mixed and heated with constant stirring. 1.26 Kg polyvinyl pyrollidone was dissolved in the above solvent with constant stirring. 8.0 Kg Acetaminophen was dissolved in the above solvent system with heating under constant stirring. The concentrated solution was encapsulated in the soft gelatin capsules.

### Example 14

TABLE 10
Composition of example 14

| Ingredients | Mg/Capsule |
|---|---|
| Paracetamol | 500.00 |
| PEG 400 | 483.00 |
| PEG 1500 | 29.00 |
| PVP K90 | 63.00 |
| Purified Water | 175.00 |
| Total weight | 1250.00 |

A solution was prepared to fill in a batch of 20,000 soft gel capsules.

0.966 Kg polyethylene glycol 400, 3.5 Kg purified water, and 0.58 Kg polyethylene glycol 1500 were mixed and heated with constant stirring. 1.26 Kg polyvinyl pyrollidone was dissolved in the above solvent with constant stirring. 10.0 Kg Acetaminophen was dissolved in the above solvent system with heating under constant stirring. The concentrated solution was encapsulated in the soft gelatin capsules.

The pharmaceutical composition which have been manufactured but didn't provide stable pharmaceutical compositions also include following:

### Group A

| | Mg per Capsule | | | | | | |
|---|---|---|---|---|---|---|---|
| Ingredients | A1 | A2 | A3 | A4 | A5 | A6 | A7 |
| Paracetamol | 325.00 | 325.00 | 325.00 | 325.00 | 325.00 | 325.00 | 325.00 |
| Pseudoephedrine HCl | 30.00 | 30.00 | 30.00 | 30.00 | 30.00 | 30.00 | 30.00 |
| Transcutol P | 90.00 | 405.00 | 70.00 | 425.00 | 335.00 | 360.00 | 355.00 |
| PEG 400 | 555.00 | 200.00 | 550.00 | 95.00 | 95.00 | 95.00 | 95.00 |
| PEG 1000 | — | — | — | 90.00 | 180.00 | 155.00 | 155.00 |
| PVP K 30 | 50.00 | 70.00 | — | 70.00 | 70.00 | 70.00 | 80.00 |
| PVP K 90 | — | — | 23.30 | — | — | — | — |
| Total wt. | 1050.00 | 960.00 | 998.30 | 1035.00 | 1035.00 | 1035.00 | 1040.00 |

### Group B

| | Mg per Capsule | | | |
|---|---|---|---|---|
| Ingredients | B1 | B2 | B3 | B4 |
| Paracetamol | 325.00 | 325.00 | 325.00 | 325.00 |
| Pseudoephedrine HCl | 30.00 | 30.00 | 30.00 | 30.00 |
| Transcutol P | 70.00 | 555.00 | 70.00 | 355.00 |
| PEG 400 | 555.00 | — | 525.00 | 200.00 |
| PEG 6000 | — | — | 10.00 | — |
| PVP K 30 | 70.00 | 70.00 | 70.00 | 70.00 |
| Purified water | — | — | 20.00 | — |
| Capryol 90 | — | — | — | 50.00 |
| Total wt. | 1050.00 | 980.00 | 1050.00 | 1030.00 |

### Group C

| | Mg per Capsule | | | | |
|---|---|---|---|---|---|
| Ingredients | C1 | C2 | C3 | C4 | C5 |
| Paracetamol | 325.00 | 325.00 | 325.00 | 325.00 | 325.00 |
| Pseudoephedrine HCl | 30.00 | 30.00 | 30.00 | 30.00 | 30.00 |
| Transcutol P | 355.00 | 380.00 | 350.00 | 350.00 | 355.00 |
| PEG 400 | 250.00 | 95.00 | 95.00 | 95.00 | 95.00 |
| PEG 1000 | — | — | — | — | 155.00 |
| PVP K 30 | 70.00 | 70.00 | 70.00 | 70.00 | 70.00 |
| NaOH | 5.00 | 5.00 | 5.00 | 5.00 | — |
| Tween | — | — | 5.00 | 5.00 | — |
| Polyvinyl alcohol | — | — | — | | 5.00 |
| Total wt. | 1035.00 | 905.00 | 880.00 | 880.00 | 1035.00 |

### Group D

| | Mg per Capsule | |
|---|---|---|
| Ingredients | D1 | D2 |
| Paracetamol | 400.00 | 500.00 |
| Transcutol P | — | 750.00 |
| PEG 400 | 455.00 | 320.00 |
| PEG 1000 | 75.00 | — |
| PVP K 30 | 100.00 | 100.00 |
| Purified water | 50.00 | 30.00 |
| NaOH | 0.50 | 0.50 |
| Total wt. | 1080.50 | 1700.50 |

The following examples illustrate the present invention by way of solubilizing as well as providing stable pharmaceutical composition of paracetamol in a soft gel. In each of the below mentioned examples (Examples 15-Example 18), soft gelatin capsules containing solution were clear, and the active drug included therein did not precipitate.

### Example 15

TABLE 11
Composition of Example 15

| Ingredients | Mg/Capsule |
|---|---|
| Paracetamol | 325.00 |
| Pseudoephidrine Hydrochloride | 30.00 |
| Dextromethorphan Hydrobromide | 10.00 |
| Transcutol P | 355.00 |
| Polyethylene Glycol 400 | 100.00 |
| Polyethylene Glycol 1000 | 75.00 |
| PVP K 30 | 100.00 |
| Purified Water | 50.00 |
| NaOH | 0.50 |
| Total weight | 1045.50 |

A solution was prepared to fill in a batch of 5000 capsules.

1.78 kg Transcutol P, 500 gm polyethylene glycol 400 and 375 gm polyethylene glycol 1000 were mixed and heated with constant stirring. 500 gm of polyvinyl pyrollidone K-30 was dissolved in the above solvent blend and heated with constant stirring. Acetaminophen 1625 gm, 150 gm Pseudoephedrine and 50 gm Dextromethorphan HBr were dissolved in the above solvent system with heating not more than 65° C. under constant stirring. The pH of the solution was adjusted from 6.5 to 7.2 by adding 1% sodium hydroxide solution. The concentrated solution was encapsulated in the soft gelatin capsules.

### Example 16

TABLE 12
Composition of Example 16

| Ingredients | Mg/Capsule |
|---|---|
| Paracetamol | 325.00 |
| Pseudoephidrine Hydrochloride | 30.00 |
| Dextromethorphan Hydrobromide | 10.00 |
| Doxylamine Succinate | 6.25 |
| Transcutol P | 355.00 |
| PEG 400 | 100.00 |
| PEG 1000 | 75.00 |
| PVP K 30 | 100.00 |
| Purified Water | 50.00 |
| NaOH | 0.50 |
| Total weight | 1051.75 |

A solution was prepared to fill in a batch of 5000 soft gel capsules.

1.78 kg Transcutol P, 500 gm polyethylene glycol 400 and 375 gm polyethylene glycol 1000 were mixed and heated with constant stirring. 500 gm of polyvinyl pyrollidone K-30 was dissolved in the above solvent blend and heated with constant stirring. 1625 gm Acetaminophen, 150 gm Pseudoephedrine, 50 gm Dextromethorphan HBr and 31.25 gm Doxylamine Succinate were dissolved in the above solvent system with heating not more than 65° C. under constant stirring. The pH of the solution was adjusted from 6.5 to 7.2 by adding 1% sodium hydroxide solution. The concentrated solution was encapsulated in soft gelatin capsules.

### Example 17

TABLE 13
Composition of Example 17

| Ingredients | Mg/Capsule |
|---|---|
| Paracetamol | 325.00 |
| Pseudoephedrine Hydrochloride | 30.00 |
| Chlorpheniramine Maleate | 2.00 |
| Transcutol P | 465.00 |
| PEG 400 | 100.00 |
| PEG 1000 | 75.00 |
| PVP K 30 | 100.00 |
| Purified Water | 40.00 |
| NaOH | 0.50 |
| Total weight | 1137.50 |

A solution was prepared to fill in a batch of 5000 soft gelatin capsules

1.78 kg Transcutol P, 500 gm polyethylene glycol 400 and 375 gm polyethylene glycol 1000 were mixed and heated with constant stirring. 500 gm of polyvinyl pyrollidone K-30 was dissolved in the above solvent blend and heated with constant stirring. Acetaminophen 1625 gm, 150 gm Pseudoephedrine and 10 gm Chlorpheniramine Maleate were dissolved in the above solvent system with heating not more than 65° C. under constant stirring. The pH of the solution was adjusted from 6.5 to 7.2 by adding 1% sodium hydroxide solution. The concentrated solution was encapsulated in the soft gelatin capsules.

### Example 18

TABLE 14
Composition of Example 18

| Ingredients | Mg/Capsule |
|---|---|
| Paracetamol | 325.00 |
| Pseudoephedrine Hydrochloride | 30.00 |
| Chlorpheniramine Maleate | 2.00 |
| Dextromethorphan Hydrobromide | 10.00 |
| Transcutol P | 465.00 |
| PEG 400 | 103.00 |
| PEG 1000 | 72.00 |
| PVP K30 | 100.00 |
| Purified Water | 40.00 |
| NaOH | 0.50 |
| Total weight | 1147.50 |

A solution was prepared to fill in a batch of 5000 soft gel capsules.

1.78 kg Transcutol P, 500 gm polyethylene glycol 400 and 375 gm polyethylene glycol 1000 were mixed and heated with constant stirring. 500 gm of polyvinyl pyrollidone K-30 was dissolved in the above solvent blend and heated with constant stirring. 1625 gm Acetaminophen, 150 gm Pseudoephedrine, 50 gm Dextromethorphan HBr and 10 gm Chlorpheniramine Maleate were dissolved in the above solvent system with heating not more than 65° C. under constant stirring. The pH of the solution was adjusted from 6.5 to 7.2 by adding 1% sodium hydroxide solution. The concentrated solution was encapsulated in the soft gelatin capsules.

The utility of the solvent system according to the invention is to improve the solubility and stability of sparingly soluble active drugs dissolved therein and filled in the soft gel capsules. Thus, it is possible to minimize the risk of leaking filling material in a soft gel capsule.

1. A stable pharmaceutical composition comprising a soft gelatin capsule that comprises at least one sparingly soluble active drug and a solvent system, wherein the solvent system comprises a solvent, a co-solvent, a solubilizer, a surfactant, an aqueous solution of alkali and a crystal growth inhibitor.

2. The composition of claim 1 wherein the at least one sparingly soluble active drug is an antipyretic or analgesic drug.

3. The composition of claim 2 wherein the antipyretic or analgesic drug is Paracetamol (Acetaminophen).

4. The composition of claim 1 further comprising a freely soluble drug, wherein the freely soluble drug is selected from an antihistamine, an antitussive, and a decongestant.

5-10. (canceled)

11. The composition of claim 4 wherein the antihistamine is selected from chlorpheniramine maleate and doxaylamine succinate; wherein the antitussive is dextromethorphan hydrobromide; and wherein the decongestant is selected from pseudoephedrine hydrochloride and phenylephrine hydrochloride.

12. The composition of claim 1 wherein the solvent of the solvent system comprises Diethylene glycol mono ethyl ether (Transcutol P).

13. The composition of claim 1 wherein the cosolvent of the solvent system comprises a combination of polyethylene glycols (PEGs), wherein the combination of PEGs comprises at least one PEG having a molecular weight below 800 dalton and at least one PEG having a molecular weight above 800 dalton.

14. The composition of claim 1 wherein the solubilizer of the solvent system is selected from polyvinyl pyrrolidone, Macrogol 15 Hydroxystearate (Solutol), Propylene Glycol Caprylate (Capryol), and Polyoxyl 40 Hydrogenated Castor Oil (Acrysol).

15. The composition of claim 1 wherein the surfactant of the solvent system is selected from Sodium Lauryl Sulphate (SLS), Propylene Glycol Caprylate (Capryol 90), Polysorbate 20 (Tween 20), and Polysorbate 80 (Tween 80).

16. The composition of claim 1 wherein the crystal growth inhibitor of the solvent system is selected from Polyvinyl Alcohol, Gelatin, Mannitol, Sodium Carboxymethyl Cellulose (CMCNa), and Povidone.

17. The composition of claim 1 wherein the alkali of the solvent system is selected from sodium hydroxide and potassium hydroxide.

18. A process for preparing a stable gelatin capsule dosage form of at least one sparingly soluble active drug comprising the steps of:
a. solubilizing the at least one sparingly soluble active drug in a solvent system, wherein the solvent system comprises a solvent, a co-solvent, a solubilizer, a surfactant, an aqueous solution of alkali and a crystal growth inhibitor; and
b. encapsulating the solution of the at least one sparingly soluble active drug in the solvent system in soft gelatin shell dosage form.

19. The process of claim 18 wherein the at least one sparingly soluble active drug is an antipyretic or analgesic drug.

20. The process of claim 19 wherein the antipyretic or analgesic drug is Paracetamol (Acetaminophen).

21. The process of claim 18 wherein the stable soft gelatin capsule dosage form further comprises a freely soluble drug, and wherein the freely soluble drug is selected from an antihistamine, an antitussive, and a decongestant.

22. The process of claim 21 wherein the antihistamine is selected from chlorpheniramine maleate and doxaylamine succinate; wherein the antitussive is dextromethorphan hydrobromide; and wherein the decongestant is selected from pseudoephedrine hydrochloride and phenylephrine hydrochloride.

23. The process of claim 18 wherein the solvent of the solvent system comprises Diethylene glycol mono ethyl ether (Transcutol P).

24. The process of claim 18 wherein the cosolvent of the solvent system comprises a combination of polyethylene glycols (PEGs), wherein the combination of PEGs comprises at least one PEG having a molecular weight below 800 dalton and at least one PEG having a molecular weight above 800 dalton.

25. The process of claim 18 wherein the solubilizer of the solvent system is selected from polyvinyl pyrrolidone, Macrogol 15 Hydroxystearate (Solutol), Propylene Glycol Caprylate (Capryol), and Polyoxyl 40 Hydrogenated Castor Oil (Acrysol).

26. The process of claim 18 wherein the surfactant of the solvent system is selected from Sodium Lauryl Sulphate (SLS), Propylene Glycol Caprylate (Capryol 90), Polysorbate 20 (Tween 20), and Polysorbate 80 (Tween 80).

27. The process of claim 18 wherein the crystal growth inhibitor of the solvent system is selected from Polyvinyl Alcohol, Gelatin, Mannitol, Sodium Carboxymethyl Cellulose (CMCNa), and Povidone.

28. The process of claim 18 wherein the alkali of the solvent system is selected from sodium hydroxide and potassium hydroxide.
